(19) United States
(12) Patent Application Publication
(10) Pub. No.: US 2025/0107714 A1
(43) Pub. Date: Apr. 3, 2025

(54) MULTIMODAL IMAGING GUIDED NONCONTACT VITAL SIGNS MONITORING SYSTEMS AND METHODS

(71) Applicant: Welch Allyn, Inc., Skaneateles Falls, NY (US)

(72) Inventors: John A. Lane, Venice, FL (US); David E. Quinn, Auburn, NY (US); Tyson B. Whitaker, Arden, NC (US); Gene Wolfe, Pittsford, NY (US); WonKyung McSweeney, Manlius, NY (US)

(21) Appl. No.: 18/830,354

(22) Filed: Sep. 10, 2024

Related U.S. Application Data
(60) Provisional application No. 63/541,638, filed on Sep. 29, 2023.

(51) Int. Cl. A61B 5/00; G16H 10/60

(52) U.S. Cl. CPC A61B 5/0059; A61B 5/6887; A61B 5/7221; G16H 10/60; A61B 2562/0204

(57) ABSTRACT

This disclosure is directed toward systems and methods for monitoring patients in a clinical setting by using non-contact multimodal imaging. For example, based on receiving imaging data, a computing device may identify an object and a region of focus formed by the object. The computing device may then cause a sensor to capture first sensor data at a first time and second sensor data at a second time, wherein the sensor data is associated with the region of focus. Based on determining that a difference between the first sensor data and the second sensor data is greater than or equal to a threshold difference, the computing device may generate and send, to an additional computing device, an alert indicative of the second sensor data.

### RELATED APPLICATION

This application claims priority to U.S. Provisional Application No. 63/541,638, filed Sep. 29, 2023, the disclosure of which is incorporated herein by reference and for all purposes.

### TECHNICAL FIELD

This application is directed to systems and methods for monitoring patents in a caregiver setting.

### BACKGROUND

As technology continues to advance, healthcare establishment devices provide increased functionality. In-patient monitoring, for example, utilizes video monitoring to assist in providing patient care. In such instances, a patient is typically monitored in a healthcare setting, such as an intensive care unit (ICU) or medical surgical room, with a fixed or mobile camera. The video feed taken by the camera may analyzed to determine a myriad of patient statistics, such as bed exiting, activities, medication diversion, and the like. In other examples, video feedback may be used to determine specific measurements associated with a patient, such as heartrate and respiratory rate. However, the use of video imaging data to derive vital signs is much less reliable. Not only do most analyzing algorithms require the patient to be stationary while the video is being analyzed, but external factors such as lighting and presentation often result in inaccurate or incomplete results.

The various example embodiments of the present disclosure are directed toward overcoming one or more of the deficiencies associated with patient management systems.

### SUMMARY

As described above, video monitoring systems may be advantageous for patient monitoring in healthcare settings. For example, traditional healthcare settings rely on healthcare professionals, such as nurses and doctors, to be physically present with a patient to collect patient information. This information ranges from behavior patterns (e.g., bed exiting, falls, sleep, medication diversion, activities, etc.) to physical measurements (e.g., heart rate, respiratory rate, oxygen saturation, etc.). This physical collection of information is extremely time consuming and can be burdensome for healthcare workers who are responsible for large numbers of patients. Moreover, because healthcare workers typically cannot monitor patients 24/7, measurements may sometimes be overlooked or even missed. A solution presents itself in video monitoring systems.

Video monitoring systems have become increasingly well-known and commonly used in healthcare settings. Typically, a patient is monitored with a fixed or mobile camera, which sends real-time video feed to staff and/or artificial intelligence (AI) systems to review. In the instance of AI, the video feed is typically input into a classification engine which may alert a healthcare worker to certain events detected in the video feed, such as a patient fall. However, video monitoring systems are not limited to behavior patterns—they may be used to detect physical measurements as well.

However, the current video monitoring systems are not without limitation. For example, existing video monitoring techniques require a myriad of factors to be in place in order to obtain accurate results. For example, in order to detect physical measurements, such as vital signs, patients must remain stationary and in full view of the camera, forcing patients to remain contained to a “monitoring area.” Moreover, AI algorithms used to determine vital signs require immutable environmental factors, such as lighting and camera angulation. However, most clinical settings are dynamic and ever-changing, resulting release-of-information occlusions, and adverse lighting, post, orientation, motion—all of which can cause imaging processing systems to fail to derive a reliable signal. The systems and methods described herein provide for accurate charting and recording of data from the multi-modal system when the data is determined to be reliable based on the multi-modal sensor data gathered regarding the patient. In this manner, the system may chart patient data to an electronic medical record in a reliable way without requiring human intervention and ensuring accuracy and quality of the charted data such that the electronic medical record may be used by caregivers for determining treatment procedures.

Thus, this application is directed towards systems and methods for monitoring patients in a clinical setting by using non-contact multimodal imaging. For example, a care facility, such as a clinic or a hospital setting, may include an imaging device and/or a sensor. The imaging device may include any device having imaging capabilities, such as a visible camera; an infrared camera; or a red, blue green (RGB) camera, to name a few non-limiting examples. In some examples, the imaging device may include image-altering feature such as pan, tilt, and zoom. The sensor may include any sensing device capable of determining one or more measurement associated with a patient. For example, the sensor may be configured to monitor vital signs and may include, for example, millimeter (mm) wave sensors or light detection and ranging (LIDAR) sensors. It may be noted that although this application describes a single imaging device and a single sensor being used, any number of imaging devices and/or sensors may be utilized herein.

In some examples, the imaging device and/or the sensor may be coupled to a moveable platform, such as a gimbal. For example, the gimbal may be capable of mechanic movement such that the imaging device and/or the sensor may be positioned to obtain a complete and accurate image of an environment in which the imaging device and/or the sensor are located. For example, the gimbal may mechanically tilt vertically or horizontally such that the imaging device and/or the sensor may obtain accurate data regarding all areas of the environment. The moveable platform may be configured to be steerable in at least one of an x-direction, a y-direction, or a z-direction within the care facility or other space.

In some aspects, the techniques described herein relate to a method, including: receiving, from a first sensor located in a care facility, first data associated with the care facility and a patient of the care facility and determining, based at least in part on the first data and by providing the first data to a machine learning model trained using data labeled with location data associated with an object in a space, a region of focus within the care facility. The method further includes causing, by a computing device of the care facility, a second sensor located in the care facility to capture second data associated with the region of focus at a first time and determining, based at least in part on the first data or the second data, a physiological parameter associated with the patient. The method additionally includes determining, based at least in part on the second data and the first data, a confidence score associated with the physiological parameter and recording, based at least in part on the confidence score, the physiological parameter in association with an electronic medical record associated with the patient.

### BRIEF DESCRIPTION OF THE DRAWINGS

FIG. 1 illustrates a schematic block diagram of an example patient management system environment.

FIG. 2 illustrates a top-view of an example patient management system environment.

FIG. 3 shows a schematic block-diagram of an example process for providing an alert to a clinician device indicating that a patient is in need of caretaker intervention based on sensor data.

FIG. 4 illustrates an example process for alerting a clinician device that a patient may have fallen or is a fall risk.

FIG. 5 illustrates an example process for monitoring patient characteristics using infrared cameras and RGB cameras based on lighting scenarios within the patient room.

FIG. 6 illustrates an example process for monitoring patient characteristics and alerting a clinician device to indicate that a patient is in need of caretaker intervention based on sensor data

FIG. 7 is an example computing system and device which may be used to implement the described techniques.

### DETAILED DESCRIPTION

Systems and methods disclosed and contemplated herein are directed towards monitoring patient vital signs using non-contact multimodal imaging. Various embodiments of the present disclosure will be described in detail with reference to the drawings, wherein like reference numerals represent like parts and assemblies throughout the several views. Additionally, any examples set forth in this specification are not intended to be limiting and merely set forth some of the many possible embodiments.

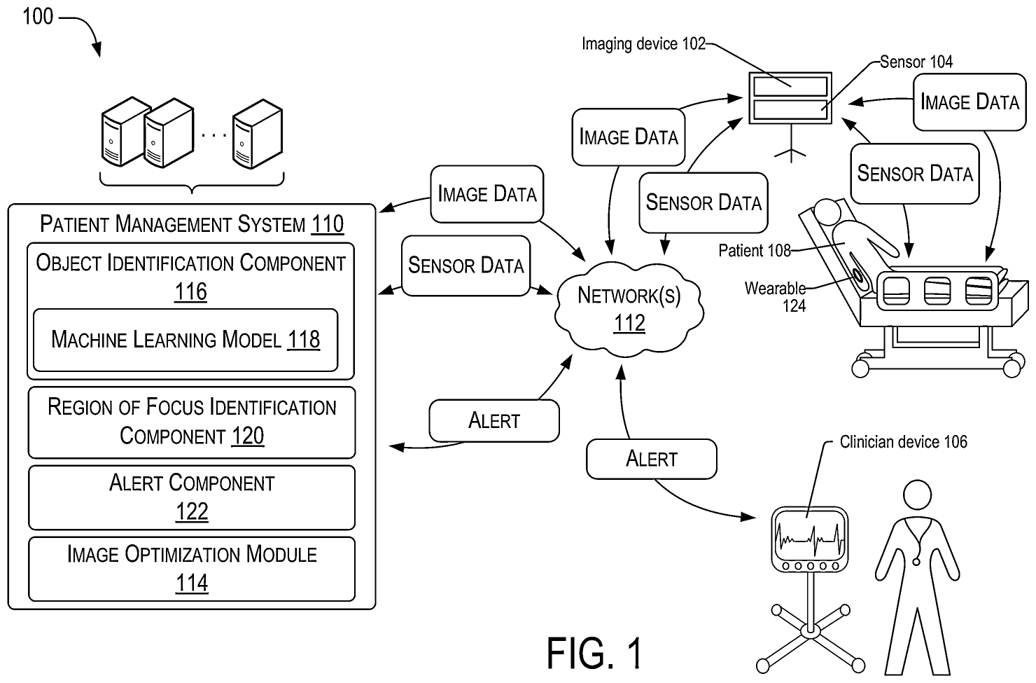

FIG. 1 shows a schematic block diagram of an example patient management system environment 100 used to monitor patient characteristics and alert clinicians that the patient may be in need of assistance or intervention based on the patient characteristics being outside a threshold range. The example patient management system environment 100 includes an imaging device 102, a sensor 104, a clinician device 106, a patient management system 110, and a patient 108. The imaging device 102, the sensor 104, the clinician device 106, and/or the patient management system 110 may be in communication via one or more networks 112.

In some examples, the imaging device 102 may include any device having imaging capabilities enabling the imaging device 102 to locate an object in the environment, such as a healthcare setting. For example, the imaging device 102 may include a camera, such as an infrared camera, an RGB camera, a thermal camera, or other such imaging device to name a few non-limiting examples. In some examples, the imaging device 102 may include a device capable of capturing still images. Additionally or alternatively, the imaging device 102 may include a video camera which may be capable of capturing a stream of imaging data for continuous monitoring. In some examples, the imaging device 102 may be utilized to determine a location of an object located within the healthcare environment, such as a hospital or a clinic. Oftentimes, the object being located may be a patient, such as patient 108; thus, the imaging device 102 may be configured to determine the location of the patient 108. However, in other examples, the object may include one or more items that is not the patient 108, but which is associated with the patient 108 and indicative of a characteristic of the patient 108. For example, this may include a wearable 124 or medical device associated with the patient 108 such as wristband, patch, a heartrate monitor, a blood pressure cuff, or an intravenous fluid (IV) bag.

In some examples, the example patient management system environment 100 may include at least one or more sensors, such as sensor 104. The sensor 104 may include any device capable of non-contact monitoring of the patient 108 to determine one or more characteristics associated with the patient 108. In some examples, the sensor 104 may be utilized to determine a characteristic associated with the patient 108. For example, the sensor 104 may be utilized to monitor one or more vital signs of a patient 108, such as a respiratory rate of the patient 108 or a heartrate of the patient 108. To monitor the respiratory rate of the patient 108, for example, a RGB camera may be used. For example, the RGB camera may detect a rise and fall of the chest of the patient 108 over a period of time by, via image processing techniques, analyzing pixel intensity changes to yield a sinusoidal waveform indicating the respiratory pattern of the patient 108. However, utilizing an RGB camera as a sensor is merely one example, and any type of sensing device may be used. For example, the sensing device may include an imaging device, such as an IR camera or an IR camera. In other examples, the sensor may be specific to determine a patient characteristic, such as a patient sensing device that is capable of measuring patient parameters and/or data including pulse rate measurement sensors, blood oxygenation sensors, temperature sensors, or other such sensors. In some examples, the sensors may be positioned within the room adjacent the patient 108 and/or worn by the patient 108. Various non-limiting examples of sensors are described herein.

In some examples, the imaging device 102/or the sensor 104 may be coupled to a gimbal or steerable structure which may enable the imaging device 102 and/or sensor 104 to mechanically rotate around one or more axis (e.g., x, y, z), providing the imaging device 102 and/or the sensor 104 stability with fine control of the active area and/or field of view as it obtains image data and sensor data. Moreover, in some examples, one or more axes of the gimbal may be locked such that the imaging device 102 and/or the sensor 104 is constrained to movement in a direction of an axis. Thus, the imaging device 102 and/or the sensor 104 may obtain a 360-degree view of the environment. In some examples, the sensors of the system may be facing the same direction and/or facing different directions in different embodiments. In some examples, the different sensors of the system may each have a corresponding gimbal and/or steerable structure that allows independent positioning of the sensors (e.g., to have varying fields of view) as determined by a control system. In some examples, the sensors may be connected to the gimbal(s) and/or steerable structures such that one sensor may act as a primary sensor with one or more other sensors following the field of view as secondary sensors. For instance, a primary sensor such as a camera may be steered to have a particular field of view and a secondary sensor such as a radar device may be steered such that an active sensing region of the radar device overlaps or coordinates with the field of view of the camera. In some examples the primary sensor and the secondary sensors may overlap and/or be directed to the same area or field of view. In some examples, the primary sensor and the secondary sensors may be directed to different fields of view to provide coverage over a broader area within the care facility, e.g., such that the fields of view of the sensors are partially overlapping or non-overlapping but are adjacent one another.

In examples, the clinician device 106 may include a computing device such as a mobile phone, a tablet computer, a laptop computer, a desktop computer, and so forth which may provide a clinician (e.g., a doctor, nurse, technician, pharmacist, dentist, etc.) with information about the health of the patient 108. In some cases, the clinician device 106 may exist within a healthcare establishment, although examples are also considered in which the clinician device 106 exists and/or is transported outside of a healthcare establishment, such as a doctor's mobile phone or home desktop computer that the doctor may use when the doctor is on-call. In some examples, the clinician device 106 may include a processor, microprocessor, and/or other computing device components, shown and described below.

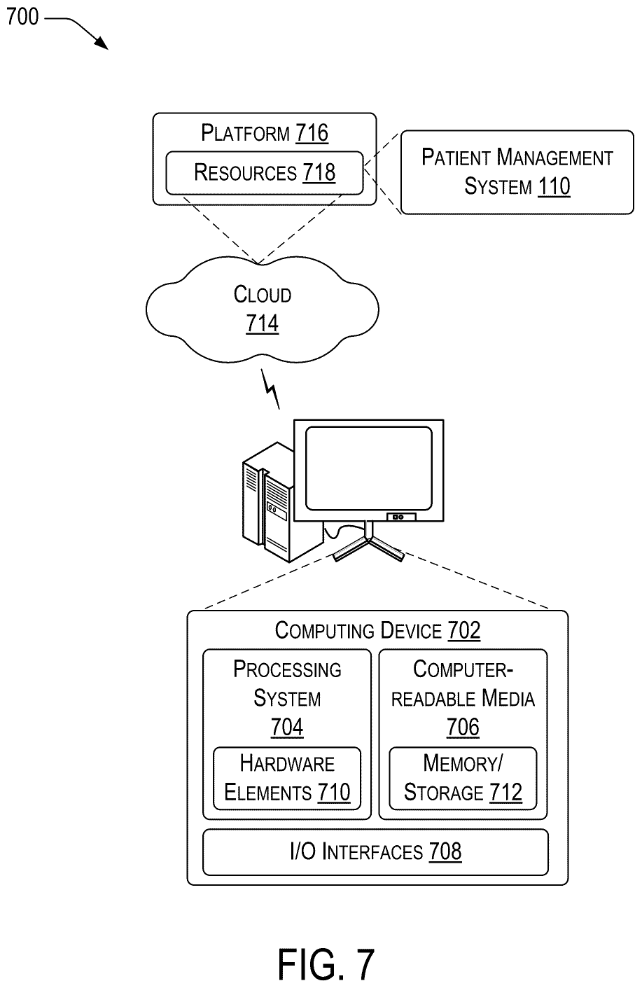

The example patient management system environment 100 may include a patient management system 110 which may be comprised of one or more server computing devices, and which may communicate with the imaging device 102 and the sensor 104 to respond to queries, receive data, respond to data, and so forth. Communication between the patient management system 110, the imaging device 102, the sensor 104, and/or the clinician device 106 occurs via the network 112 where the communication can include imaging data, sensor data, and/or patient data related to the health of the patient. A server of the patient management system 110 may act on these requests from the imaging device 102, the sensor 104, and/or the clinician device 106 and determine one or more responses to these queries, and respond back to the imaging device 102, the sensor 104, and/or the clinician device 106. A server of the patient management system 110 may also include one or more processors, microprocessors, or other computing devices as discussed in more detail in relation to FIG. 7.

The patient management system 110 may include one or more database systems accessible by a server storing different types of information. For instance, a database can store correlations and algorithms used to manage the imaging data, signal data, and other patient data to be shared between the imaging device 102, the sensor 104, and/or the clinician device 106. A database can also include clinical data. A database may reside on a server of the patient management system 110 or on separate computing device(s) accessible by the patient management system 110.

The network 112 is typically any type of wireless network or other communication network known in the art. Examples of the network 112 include the Internet, an intranet, a wide area network (WAN), a local area network (LAN), and a virtual private network (VPN), cellular network connections and connections made using protocols such as 802.11a, b, g, n and/or ac. Alternatively or additionally, the network 112 may include a nanoscale network, a near-field communication network, a body-area network (BAN), a personal-area network (PAN), a near-me area network (NAN), a campus-area network (CAN), and/or an inter-area network (IAN).

In some examples, the patient management system 110, the imaging device 102, the sensor 104, and/or the clinician device 106 may generate, store, and/or selectively share signals, imaging data, sensor data, and/or other patient data between one another to provide the patient and/or clinicians treating the patient with improved outcomes by accurately monitoring the patient characteristics and alerting clinicians when a change in the characteristics of the patient may indicate that the patient is in need of caretaker intervention.

For example, the imaging device 102 may capture image data associated with a patient in a healthcare facility and send the image data to the patient management system 110. In some examples, capturing the image data may be in response to a request, such as by a clinician at the patient management system 110, to determine and/or monitor a characteristic associated with the patient. The characteristic may include any number of measurable metrics associated with a patient, such as vital signs (e.g., a body temperature of the patient, a pulse rate of the patient, a respiratory rate of the patient, a blood pressure of the patient, etc.), intake and output (e.g., fluid intake and fluid outtake, medicine intake, etc.), or a movement of the patient, to name a few non-limiting examples. While some characteristics may be measured by monitoring a patient directly (e.g., respiratory rate), other characteristics may be measured by monitoring objects associated with the patient, such as a catheter or an IV fluid bag. Thus, the image data may represent an object, wherein the term “object” may not only refer to a patient directly, but any object that may be associated with the patient which may be indicative of a characteristic of the patient 108.

In some examples, the patient management system 110 may process the image data to optimize the image. For example, the patient management system 110 may input the image data into an image optimization module 114 of the patient management system 110 which may alter the image data such that the image data is optimized to be at a highest quality. For example, the image optimization module 114 may automatically assess the image data and adjust the image data to increase a resolution of the image data, re-format the image data into a correct format, re-size the image data to a correct dimension, or compress the image data, to name few non-limiting examples. Thus, by optimizing the image data, the patient management system 110 may obtain more accurate images, thereby more accurately identifying the object(s) in the image data. The image optimization performed by the image optimization module 114 may include preprocessing, segmenting, and corrections. During the preprocessing stage, input sensor data may be corrected for defective pixels using an input lookup table and may also undergo contrast expansion using a dynamic range histogram approach and may also undergo color correction for white balance and other illumination present in the environment based on global image data of the environment. During the segmentation stage, the input image data is processed using image processing techniques and morphology operations to localize objects, people, structures, and other such items as regions within the preprocessed image data. During the segmentation stage, particular regions that require special corrections, such as those illuminated by super bright LEDs, fluorescent light fixtures, dark corners, and other such regions may be identified and segmented. During the correction stage, the identified segments may be adjusted by lowering or raising the dynamic range of the segment and/or by applying a custom color lookup table to adjust the image data in the segments.

In some examples, the patient management system 110 may determine the object. For example, based on capturing the image data, the imaging device may send the image data to an object identification component 116 of the patient management system 110. In some examples, the object identification component 116 may include a machine learning model 118 trained to identify one or more objects in image data. For example, the machine learning model 118 may include an artificial neural network, a decision tree, a regression algorithm, or other machine learning algorithm to determine one or more objects in image data. The machine learning model 118 may be trained using training data including other image data including one or more objects. Using the training data, the machine learning model 118 may be trained to detect and/or identify objects within the image data. Moreover, the machine learning model 118 may use image data previously imputed into the machine learning model to continue to train the machine learning model, thus increasing the accuracy of the machine learning model.

The object identification component 116 may also interact with the imaging device 102 to alter the image data and improve confidence in the object detection. For example, the focus and/or lighting settings, zoom, and other imaging characteristics maybe adjusted based on the object detection. Also, if an object is partially visible, the imaging device 102 may be rotated or moved to pan or tilt towards it. If a target object is not visible in the image data, the imaging device 102 may be moved to search for the object within the room. In some examples, additional training data may be supplied to the machine learning model 118 to provide labels to image data indicative of partial and/or undetected objects in the image data. The machine learning model 118 may be used to determine confidence and/or quality of image data for determination of objects. For example, the machine learning model 118 may be used to determine image quality and/or characteristics and create additional image data (e.g., by controlling the imaging device 102 to improve the image data and/or by detecting partial or missing objects).

Based on determining the object within the image data, a region of focus identification component 120 of the patient management system 110 may identify a region of focus formed by the object. The region of focus may include a “target” region in which a sensor may more accurately acquire sensor data associated with the object. For example, a region of focus for a sensor determining a respiratory rate of a patient may be a chest of the patient. Alternatively, a region of focus for determining a liquid output of a patient may be a catheter of the patient. In some examples, the region of focus identification component 120 may determine the region of focus based in part on the request to determine and/or monitor a characteristic associated with the patient. For example, based on receiving the request, the patient management system 110 may determine a region of interest associated with the object which is likely to be associated with the request. Additionally or alternatively, in some examples, the region of focus identification component 120 may determine the region of focus based on a detection of the sensor 104. For example, the region of focus identification component 120 may determine sensor data that is most likely to be captured by the sensor 104. Based on the determination of the sensor data that is most likely to be captured by the sensor 104, the patient management system 110 may determine a ref focus that is most likely to be associated with the sensor data. Moreover, in some example, multiple objects and/or multiple regions of focus may be identified. For example, a region of focus for determining a patients fluid input and output may be both an IV and a catheter associated with the patient.

Based on identifying the region of focus, the patient management system 110 may cause the sensor 104 to capture sensor data associated with the region of focus. Sensor data may include any data received with the sensor 104 and associated with the object. This can include, for example, a measure of light, temperature, movement, pressure, speed, proximity, or humidity, to name a few non-limiting examples. In some examples, the sensor 104 may capture sensor data at various times to determine the characteristic associated with the patient. Continuing with the example above in which the characteristic is the respiratory rate of a patient, sensor data may be determined at various times in order to accurately determine the respiratory rate of the patient. For example, the sensor 104 may capture first sensor data at a first time, wherein the first sensor data indicates a first rise and fall of a chest of the patent. The sensor 104 may then capture second sensor data at a second time, wherein the second sensor data may indicate a second rise and fall of the chest of the patent. Based on determining a period of time from the first time to the second time, the patient management system 110 may determine the respiratory rate of the patient.

In some examples, sensor 104 used may be determined based on the image data. For example, the type of sensor used may be based at least in part on the image data captured by the imaging device 102 and/or sensor 104. Based on capturing the image data, the sensor 104 and the associated patient management system 110 may determine one or more environmental attributes associated with a location associated with the imaging device 102. This an include factors such as lighting and temperature, to name a few examples. Based on the request to determine the characteristic associated with the patient 108, the patient management system 110 may determine one or more sensors which may be suitable based on the environmental factor(s) such that the sensor 104 may be optimized for collecting sensor data for the respective environment which the sensor 104 is located.

In some examples, sensor data collected at multiple times may be utilized to determine that the patient requires or is likely to require caretaker intervention. For example, based on receiving first sensor data and second sensor data, an alert component 122 of the patient management system 110 may determine a difference between the first sensor data and the second sensor data. The alert component 122 may compare the difference between the first sensor data and the second sensor data. In some examples, a difference between the first sensor data and the second sensor data may be indicate a normal fluctuation associated with a condition. For example, a slight variation in a respiration rate of a patient may be normal and may not be a cause for concern. However, a larger variation may indicate a change in a condition of the patient, which may require intervention by a clinician. For example, the alert component 122 may determine that the difference between the first sensor data and the second sensor data is greater than or equal to a threshold difference between the first sensor data and the second sensor data. The threshold difference may be specific to the condition and/or the patient, such that a determination that the difference between the first sensor data and the second sensor data is greater than or equal to the threshold difference may accurately indicate that the patient is likely to require assistance or intervention. In some examples, the various thresholds described herein may be customizable and/or adjustable based on user inputs and/or previous patient data.

In some examples, based on a determination that the threshold difference between the first sensor data and the second sensor data is greater than or equal to the threshold difference, the alert component 122 may generate an alert indicative of the second sensor data. Alert component 122 may then send the alert, including the second sensor data, to the clinician device 106, thereby alerting the clinician that the patient may require intervention or assistance. For example, the alert may appear automatically on the clinician device as a pop-up notification, indicating to the clinician that the patient may be in need of assistance or intervention.

In some examples, the first sensor data may be used to determine one or more patient physiological parameters and the second sensor data may be used to determine a confidence level in the first sensor data, such as to determine when to add the determine physiological parameter to the electronic medical record of the patient. In an example, the imaging device 102 may be used to determine a physiological parameter, and based on the image characteristics and/or confidence output by the machine learning model 118, the system 100 may determine to use sensor 104 to gather additional sensor data for use in determining whether to add the physiological parameter to the electronic medical record of the patient 108. In some instances, the sensor 104 may be used to determine a position or motion of the patient or other data regarding the patient. In an illustrative example, a respiratory rate or pulse rate may be determined based on the data from the imaging device 102. The sensor 104, which may include a radar device in the illustrative example, may be used to determine the position of the patient 108 and/or if the patient 108 is moving during determination of the respiratory rate or pulse rate. In the event that the patient is moving, the confidence in the physiological parameter may be reduced. In the event that the patient 108 is stationary (and perhaps has been stationary for a threshold period of time) the system 100 may increase the confidence in the physiological parameter as reflective of the true state of the patient and may determine to chart or record the physiological parameter to the electronic medical record of the patient 108 based on the confidence score.

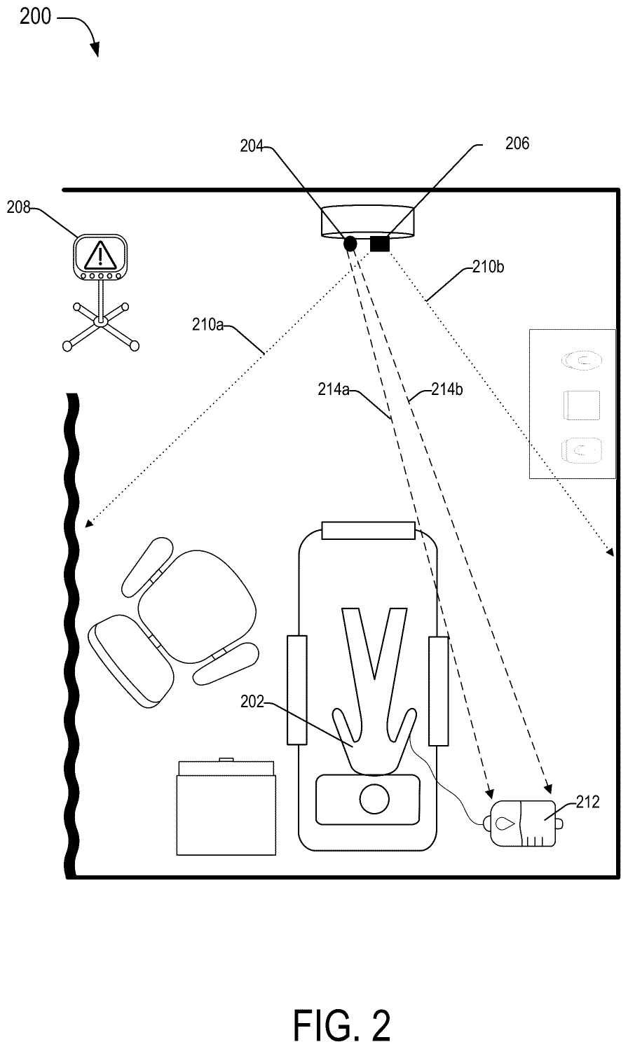

Example configurations of the imaging device 102, the sensor 104, the clinician device 106, and methods for their use, are shown and described with reference to at least FIGS. 2-6 below.

FIG. 2 illustrates a top-view of an environment 200 for a patient management system. For example, the environment 200 is illustrated as a hospital room housing a patient 202. However, this application anticipates the environment 200 being any healthcare setting in which a patient may be observed, such as an operating room, an outpatient facility, a clinical lab, to name a few non-limiting examples. The environment 200 may include an imaging device 204 and a sensor 206, similar to imaging device 102 and the sensor 104, described above with respect to FIG. 1. The environment 200 may additionally include a clinician device 208, which may be the same as or similar to the clinician device 106.

In some examples, the imaging device 204 may obtain image data of the environment 200. For example, illustrated by the dotted lines 210a and 210b, the imaging device 204 may scan or take images of the environment 200. The imaging data may then be analyzed to determine one or more objects in the environment. As described above, an object may be one or more item which may be detected and captured by a sensor to obtain sensor data indicative of a condition associated with a patient. For example, although the object may be the patient, it may additionally or alternatively be an item associated with the patient. In the current illustration, the object includes an IV fluid bag, indicated by object 212.

Based on a determination of the object, the sensor 206 may obtain sensor data associated with the object 212. For example, while the imaging device 204 may obtain image data of the environment 200, the sensor 206 may utilize the imaging data obtained by the imaging device 204 to obtain data directed toward a portion of the environment 200. By determining specific spot for the sensor 206 to obtain sensor data, the sensor is more likely to obtain accurate sensor data. For example, illustrated by the dashed lines 214a and 214b, the sensor 206 may scan or take images of the object 212 within the environment 200.

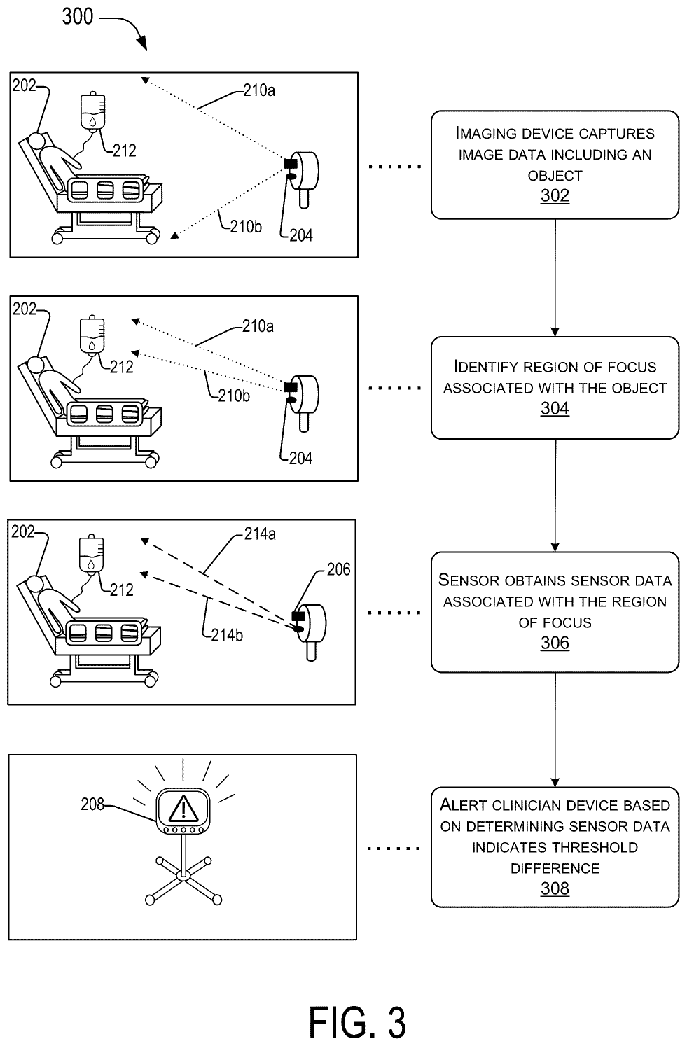

FIG. 3 shows a schematic block-diagram of a process 300 for providing an alert to a clinician device indicating that a patient is in need of caretaker intervention based on sensor data. The process 300 may include the patient 202, the imaging device 204, the sensor 206, the clinician device 208, and the object 212 illustrated in FIG. 2.

For example, at operation 302, the imaging device 204 may capture image data. For example, the imaging device 204 may be located in a healthcare facility, such as a hospital room, and may be configured to capture images of the environment which the imaging device 204 is located. The imaging device 204 may include any device capable of capturing one or more images of an environment in which the imaging device is located. For example, and as illustrated in the current embodiment, the imaging device 204 may be a RGB camera capable of detecting one or more object in an environment. For example, the imaging device 204 may be located in a room in which the patient 202 is located, and may be configured to capture one or more images of the room. For example, the current embodiment illustrates the imaging device 204 scanning the environment by dotted lines 210a and 210b to capture image data. In some examples, the imaging device 204 may send the image data to a patient management system (not illustrated) which may input the image data into a machine learning model trained to identify one or more objects in the image data. In the current illustration, the machine learning model detects the patient 202 and an IV fluid bag connected to the patient 202 as the object. In some examples, the machine learning model may detect a single object including multiple items (e.g., a patient and an associated device). In other examples, the machine learning model may detect multiple objects which may or may not be associated with another.

At operation 304, the patient management system may determine a region of focus associated with the object. In the current illustration, the patient management system detected the object 212 (the IV fluid bag) as the region of focus. A region of focus may include a “target” area in which a sensor may more accidently determine sensor data associated with the object. In some examples, the region of focus may be determined based on a request, from a clinician, to determine a characteristic associated with the patient 202. For example, a nurse may wish to monitor a fluid input level associated with the patient 202, and thus may enter, into the patient management system, a corresponding request. Based at least in part on the request, the patient management system may determine a region of focus associated with the request and/or the image data. Continuing with the current illustration, the patient management system may determine that based on the image data containing an IV fluid bag connected to the patient 202 and the request including monitoring patient fluid levels, the region of focus is the IV fluid bag.

At operation 306, a sensor, such as sensor 206, may obtain sensor data associated with the region of focus. The sensor 206 may be any sensing device which may be used to obtain sensor data associated with a patient. In the current illustration, the sensor 206 may include a thermal imaging camera which may detect variations in the levels of the fluids in the IV fluid bag. For example, to monitor fluid input levels, sensor data may be acquired at various times, such as first sensor data at a first time and second sensor data at a second time. The sensor may send the first sensor data and the second sensor data to the patient management system, which may compare the first sensor data and the second sensor data to determine one or more changes in the sensor data. In some examples, the patient management system may have a pre-programed list of threshold levels associated with various patient characteristics. In some examples, the threshold levels may be associated with a period of time. This may include, for example, a change in vital signs of a patient over a short period of time (e.g., a rapid increase or decrease in heartrate). In some examples, based on determining that the difference between the first sensor data and the second sensor data is greater than or equal to a threshold difference, the patient monitoring system may generate an alert associated with the second sensor data. For example, continuing with the current illustration, the patient management system may determine that the change in the volume of fluids that the patient 202 is intaking may be greater than a threshold value of fluid that the patient 202 should be intaking, which may be cause for intervention.

At operation 308, the patient management system may send the alert to the clinician device 208. The alert may include the second sensor data, such that the clinician receiving the alert is aware of the condition associated with the patient 202. In some examples, the alert may include additional information such as other healthcare data associated with the patient 202, a location of the patient 202, a condition of the patient 202, or a doctor assigned to the patient 202, to name a new examples. In this way, healthcare workers may be able to easily and effectively monitor patients without physically being present or physically monitoring the patient 202 themselves, and be alerted when a condition may be detected.

In some examples, at operation 308, the system may determine whether to chart or record the detected condition, location, physiological parameter, or other such data as detected by the sensors. The imaging device and sensor may be used to determine the data as well as confidence in the data for determining when to record the data to the electronic medical record of the patient. Such a determination may be made by a machine learning model trained using multi-modal sensor data (e.g., data sets including data from multiple types of sensors) tagged with confidence scores and/or determinations to chart or record or to ignore or discard the readings.

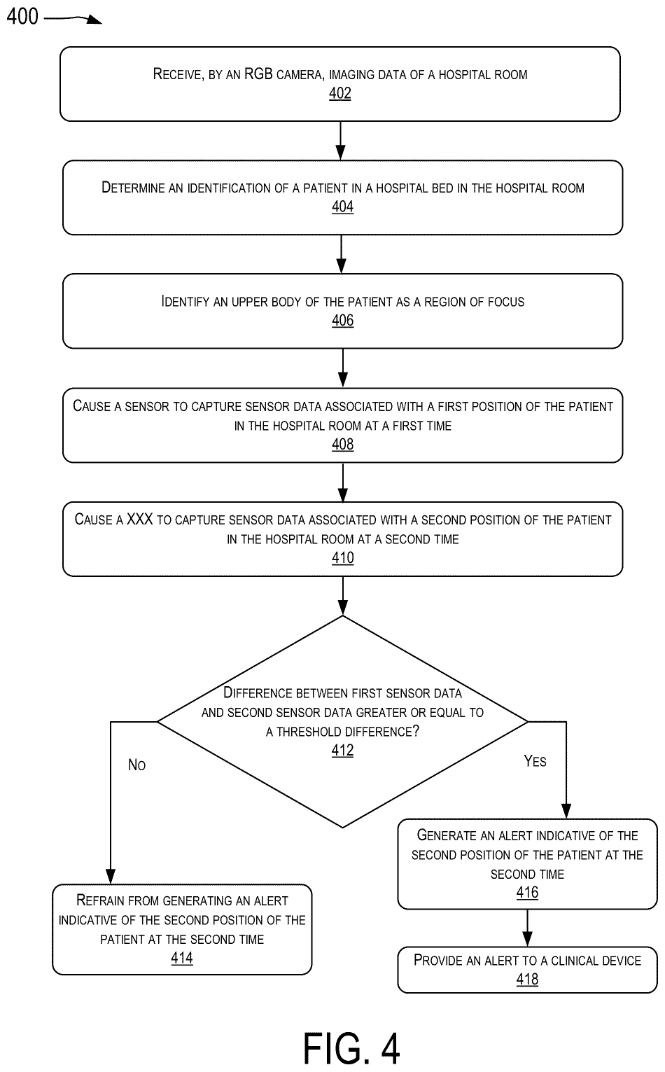

FIG. 4 illustrates a process 400 for alerting a clinician device that a patient may have fallen or is a fall risk. It may be noted that the process 400 is merely an example process to illustrate the techniques and methods described herein for utilizing one or more imaging device(s) and a sensor(s) to generate an alert associated with data captured by the sensor being greater than or equal to a threshold, thus indication that the patient may be in need in clinical assistance or intervention. As such, while the current illustration describes an RGB camera as an imaging device and a sensor, such as sensor 104, that may include any device capable of non-contact monitoring of the patient to determine one or more characteristics associated with the patient, any number and type of imaging devices and/or sensors may be used.

For example, at operation 402, the process includes receiving, by an RGB camera, image data of a hospital room. For example, a clinician or healthcare worker may wish to monitor a characteristic associated with the patient, such as a movement of the patient. For example, the patient may be a fall risk patient, and may be required to stay in the hospital bed. Rather than requiring the clinician to physically be in the hospital room to continuously monitor the patient, the RGB camera may be located in the hospital room of the patient and may be configured to continuously monitor the patient room by capturing video image data. The video data may be used to determine whether the patient has moved, which is described in detail below.

At operation 404, the process 400 may include determining an identification of the patient in the hospital room. For example, based on capturing the image data, the RGB camera may send the image data to a patient monitoring system. The patient monitoring system may include a machine learning model which may be trained and/or configured to accurately identify objects and/or regions of focus associated with image data.

At operation 406, the process 400 may include identifying an upper body of the patient as a region of focus. For example, because the location of the patient is being monitored, a movement of the upper body of the patient may indicate that the patient has moved from a desired position within the hospital bed, and may require clinician intervention. As such, the upper body of the patient may act as a sufficient region of focus in determining whether the patient has moved.

At operation 408, the process 400 may include causing a non-contact sensor patient monitoring sensor to capture sensor data associated with a first position of the patient in the hospital room at the first time. For example, the first position of the patient at the first time may serve as a baseline position. In other words, the first position of the patient at the first time may be a position in which the patient was placed in the hospital bed and is expected to remain. Similarly, at operation 410, the process may include causing the sensor to capture second sensor data associated with a second position of the patient in the hospital room at a same time after the first time.

At operation 412, the process may include determining that a difference between the first sensor data and the second sensor data is greater than or equal to a threshold difference. For example, based on receiving the sensor data, the patient management system may compare the first position to the second position to determine a difference in the position of the patient. In some examples, the threshold difference may be associated with the patient characteristic being monitored. For example, in the current illustration, because the patient is being monitored to maintain a position in the hospital bed, the threshold difference may be a small distance (e.g., 5 inches, 6 inches, 1 foot, etc.). However, the threshold difference may be any unit of measurement corresponding to the sensor data and may include any range of differences.

Thus, at operation 414 (indicated by a “No” at operation 412), the process 400 may include, based on determining that the difference between the first sensor data and the second sensor data is less than the threshold difference, refraining from generating an alert indicative of the second position of the patient at the second time. In other words, the patient management system may determine that the patient has remained in a same position, or the position that the patient has moved to is not great enough to alert a clinician.

Thus, at operation, the 414 (indicated by a “No” at operation 412), the process 400 may include, based on determining that the difference between the first sensor data and the second sensor data is less than the threshold difference, refraining from generating an alert indicative of the second position of the patient at the second time. In other words, the patient management system may determine that the patient has remained in a same position, or the position that the patient has moved to is not great enough to alert a clinician.

Alternatively, based at least in part on determining that the difference between the first sensor data and the second sensor data is greater than or equal to the threshold difference, the process 400 may include, at operation 416, generating, by the patient management system, an alert indicative of the second position of the patient at the second time. For example, the patient may have moved a distance such that the patient is now considered at risk, and may require intervention from a clinician to assist the patient back into the hospital bed. Thus, at operation 418, the process may include providing the alert to a clinician device such that a clinician may check on the patient and provide assistance if needed.

In some examples, at operation 412, the process 400 may also include determining confidence in the first sensor data and/or the second sensor data and/or the difference. In some examples the confidence may be in a physiological parameter or position of the patient. In the event that the confidence level is below a threshold, the system may attempt to gather additional sensor data using one or more sensors and/or to increase the confidence score based on the additional (e.g., second) sensor data. In some examples, rather than generating an alert, the system may determine when to record and/or chart the patient data and/or differences between the patient data based on confidence scores, as discussed herein.

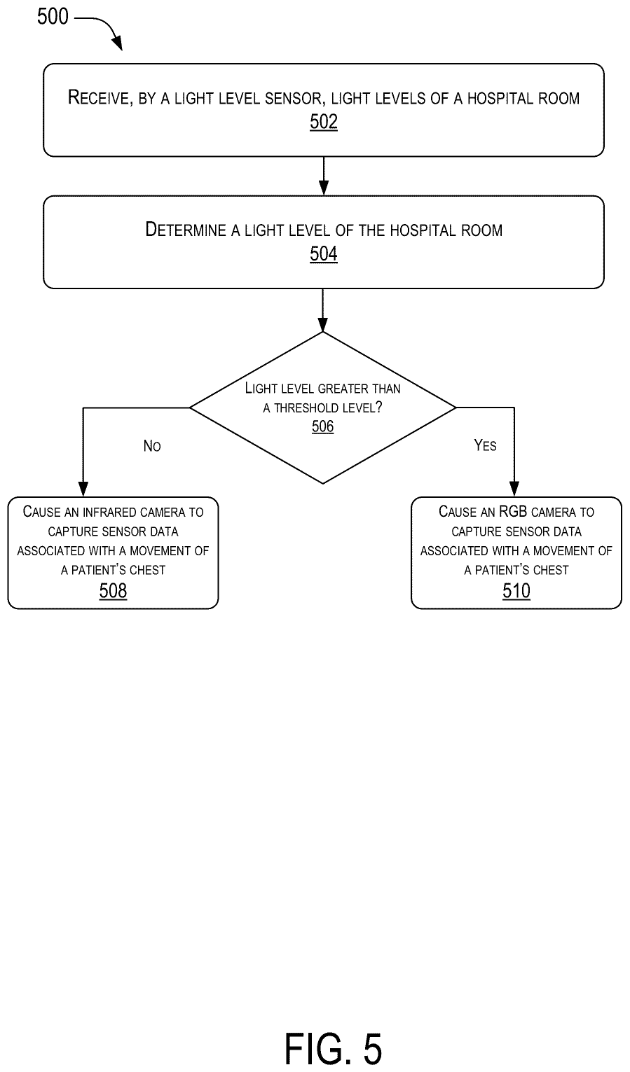

FIG. 5 illustrates a process 500 for monitoring patient characteristics using infrared cameras and RGB cameras based on lighting scenarios within the patient room. For example, at operation 502, the process 500 may include at least receiving, by a computing device (e.g., the patient management system), and by a light level sensor, light levels of a hospital room. For example, the patient management system may receive a sensor reading of ambient light within the room or an absolute brightness as read by a sensor of the room. At operation 504, the process 500 may then include the computing device determining a light level of the hospital room. The light level may be determined as a measurement of the level of ambient lighting in the room. In some examples the light level may be on a scale, such as from one to one hundred, with the low end of the scale associated with a room having no illumination and the high end of the scale associated with a room having full illumination from all light sources (e.g., light bulbs and windows etc.). The light level may be determined based on detected lumens of illumination or another similar form of measurement for light and/or brightness.

At operation 506, the computing device may determine if the light level is greater than a threshold level. The threshold may be a set value or may vary based on other sensor data or information, for example related to other factors that may impact image gathering techniques.

At operation 508, in the event that the computing device determines that the light level is below the threshold level at 506, then the computing device may cause an infrared camera to capture sensor data associated with a movement of a patient's chest. In other examples, the infrared camera (or other non-visible light camera) may be used to capture image data associated with other patient parameters, characteristics, movements, and other such data.

At operation 510, in the event that the computing device determines that the light level is above the threshold value, the computing device may cause an RGB or other visible light camera to capture sensor data associated with a movement of a patient's chest. In other examples, the visible light camera may be used to capture image data associated with other patient parameters, characteristics, movements, and other such data.

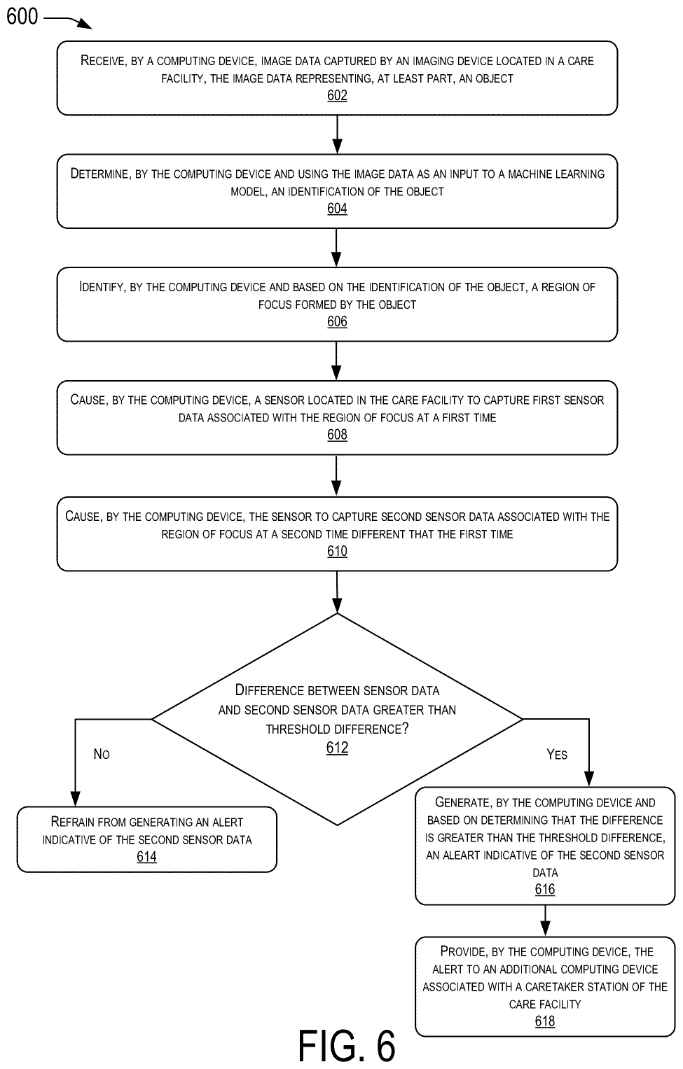

FIG. 6 illustrates a process 600 for monitoring patient characteristics to alert clinicians that a patient may be in need of assistance or intervention based on the patient characteristics being outside a threshold range of patient characteristics. For example, at operation 602, the process 600 may include at least receiving, by a computing device (also referred to herein as a “patient management system”), image data captured by an imaging device located in a care facility. In some examples, the image data may represent, at least part, an object. For example, the imaging device may be any device capable of capturing image data, such as an infrared camera, a RGB camera, a thermal camera, or a light sensing camera, to name a few non-limiting examples. In some examples, the imaging device may include a device capable of capturing still images. Additionally, or alternatively, the imaging device may include a video camera which may be capable of capturing a stream of imaging data for continuous monitoring.

At operation 604, the process 600 may include determining, by the computing device and using the image data as an input to a machine learning model, an identification of the object. For example, the machine learning model may include an artificial neural network, a decision tree, a regression algorithm, or other machine learning algorithm to determine one or more objects in image data. In some examples, the object identified by the image data may be a medical device, such as a wristband, a heartrate monitor, a blood pressure cuff, or an intravenous fluid (IV) bag, to name a few non-limiting examples. Further, the identified object may represent a patient, a physiological symptom (e.g., a body temperature of the patient, a pulse rate of the patient, a respiratory rate of the patient, a blood pressure of the patient, etc.), or an intake and output (e.g., fluid intake and fluid outtake, medicine intake, etc.), to name a few non-limiting examples. Thus, the image data may represent an object, wherein the term “object” may not only refer to a patient directly, but any object that may be associated with the patient which may be indicative of a characteristic of the patient.

At operation 606, the process 600 may include identifying, by the computing device and based on the identification of the object, a region of focus formed by the object. The region of focus may include a “target” region in which a sensor may more accurately acquire sensor data associated with the object. For example, a region of focus for a sensor determining a respiratory rate of a patient may be a chest of the patient. Alternatively, a region of focus for determining a liquid output of a patient may be a catheter of the patient. In some examples, the region of focus may be determined based in part on the request to determine and/or monitor a characteristic associated with the patient. Additionally, or alternatively, in some examples, the region of focus may be determined based on a detection by a sensor. For example, the region of focus may determine sensor data that is most likely to be captured by the sensor. Based on the determination of the sensor data that is most likely to be captured by the sensor, the computing device may determine a region of focus that is most likely to be associated with the sensor data. Moreover, in some examples, multiple objects and/or multiple regions of focus may be identified. For example, a region of focus for determining a patients fluid input and output may be both an IV and a catheter associated with the patient.

At operation 608, the process 600 may include causing, by the computing device, a sensor located in the care facility to capture first sensor data associated with the region of focus at a first time. Sensor data may include any data received with the sensor and associated with the object. This can include, for example, a measure of light, temperature, movement, pressure, speed, proximity, or humidity, to name a few non-limiting examples. In some examples, the sensor may be determined based on the image data. Based on capturing the image data, the imaging device may determine one or more environmental attributes associated with a location associated with the imaging device, such as lighting and temperature, to name a few examples. Based on the request to determine the characteristic associated with the patient, the patient management system may determine one or more sensors which may be suitable based on the environmental factor(s) such that the sensor may be optimized for collecting sensor data for the respective environment which the sensor is located.

At operation 610, the process 600 may include causing, by the computing device, the sensor to capture second sensor data associated with the region of focus at a second time different than the first time. In some examples, the sensor may capture sensor data at various times to determine the characteristic associated with the patient. Continuing with the example above in which the characteristic is the respiratory rate of a patient, sensor data may be determined at various times in order to accurately determine the respiratory rate of the patient. For example, the sensor may capture first sensor data at a first time, wherein the first sensor data indicates a first rise and fall of a chest of the patient. The sensor may then capture second sensor data at a second time, wherein the second sensor data may indicate a second rise and fall of the chest of the patent. Based on determining a period of time from the first time to the second time, the patient management system 110 may determine the respiratory rate of the patient.

At operation 612, the process 600 may include determining that the difference between the first sensor data and the second sensor data is greater than a threshold difference. For example, based on receiving first sensor data and second sensor data, an alert component of the patient management system may determine a difference between the first sensor data and the second sensor data. In some examples, a difference between the first sensor data and the second sensor data may be indicate a normal fluctuation associated with a condition. For example, a slight variation in a respiration rate of a patient may be normal and may not be a cause for concern. However, a larger variation may indicate a change in a condition of the patient, which may require intervention by a clinician. Continuing with the example above in which the characteristic is the respiratory rate, wherein the first and second sensor data indicates a first and a second rise and fall of a chest of the patient. The threshold difference may be specific to the condition and/or the patient, such that a determination that the difference between the first sensor data and the second sensor data is greater than or equal to the threshold difference may accurately indicate that the patient is likely to require assistance or intervention. The computing device may compare the first and second sensor data (i.e., the patient's rate of respiration) and determine that the rate of respiration is greater than a threshold difference/rate. In some examples, depending on the computing devices determination, the patient management system may or may not generate an alert indicative of the difference being greater than the predetermined threshold.

Based on determining that the difference between the first sensor data and the second sensor data is less than the threshold difference (e.g., “No” at operation 612), the process 600 may include, at operation 614, refraining from generating an alert indicative of the second sensor data. Continuing with the example above in which the characteristic is the respiratory rate, the computing device may determine that the rate of respiration of the patient rate is lower than the threshold respiration rate, and therefore the patient management system will refrain from generating an alert.

Alternatively, based on determining that the difference between the first sensor data and the second sensor data is greater than or equal to the threshold difference in sensor data (e.g., “Yes” at operation 612), the process 600 may include, at operation 616, generating, by the computing device, an alert indicative of the second sensor data. Continuing with the example above in which the characteristic is the respiratory rate, wherein the first and second sensor data indicates a first and a second rise and fall of a chest of the patient. The computing device may determine that the respiration rate of the patient is greater than or equal to the threshold difference in respiratory rate, and may generate an alert indicative of the difference.

At operation 618, the process may include providing, by the computing device, the alert to an addition computing device associated with a caretaker station of the care facility. In some examples, sensor data collected at multiple times may be utilized to determine that the patient requires caretaker intervention. The threshold difference may be specific to the condition and/or the patient, such that a determination that the difference between the first sensor data and the second sensor data is greater than or equal to the threshold difference may accurately indicate that the patient is likely to require assistance or intervention. Continuing with the example above in which the characteristic is the respiratory rate, the patient management system may determine the respiratory rate of the patient is greater than or equal to the threshold difference in respiratory rate and may thus send the alert, including the second sensor data, to the clinician device. For example, the alert may appear automatically on the clinician device as a pop-up notification, indicating to the clinician that the patient may be in need of assistance or intervention.

### Example System and Device

FIG. 7 illustrates an example system generally at 700 that includes a computing device 702 that is representative of one or more computing systems and/or devices that may implement the various techniques described herein. This is illustrated through inclusion of the patient management system 110. The computing device 702 may be, for example, a server of a service provider, a device associated with a client (e.g., a client device), an on-chip system, and/or any other suitable computing device or computing system.

The computing device 702 as illustrated includes a processing system 704, one or more computer-readable media 706, and one or more I/O interface 708 that are communicatively coupled, one to another. Although not shown, the computing device 702 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 704 is representative of functionality to perform one or more operations using hardware. Accordingly, the processing system 704 is illustrated as including hardware element 710 that may be configured as processors, functional blocks, and so forth. This may include implementation in hardware as an application specific integrated circuit or other logic device formed using one or more semiconductors. The hardware elements 710 are not limited by the materials from which they are formed or the processing mechanisms employed therein. For example, processors may be comprised of semiconductor(s) and/or transistors (e.g., electronic integrated circuits (Ics)). In such a context, processor-executable instructions may be electronically-executable instructions.

The computer-readable media 706 is illustrated as including memory/storage component 712. The memory/storage component 712 represents memory/storage capacity associated with one or more computer-readable media. The memory/storage component 712 may include volatile media (such as random access memory (RAM)) and/or nonvolatile media (such as read only memory (ROM), Flash memory, optical disks, magnetic disks, and so forth). The memory/storage component 712 may include fixed media (e.g., RAM, ROM, a fixed hard drive, and so on) as well as removable media (e.g., Flash memory, a removable hard drive, an optical disc, and so forth). The computer-readable media 706 may be configured in a variety of other ways as further described below.

I/O interface 708 (Input/Output interface) are representative of functionality to allow a user to enter commands and information to computing device 702, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 702 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” “logic,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on and/or transmitted across some form of computer-readable media. The computer-readable media may include a variety of media that may be accessed by the computing device 702. By way of example, and not limitation, computer-readable media may include “computer-readable storage media” and “computer-readable transmission media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer-readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable transmission media” may refer to a medium that is configured to transmit instructions to the hardware of the computing device 702, such as via a network. Computer-readable transmission media typically may transmit computer-readable instructions, data structures, program modules, or other data in a modulated data signal, such as carrier waves, data signals, or other transport mechanism. Computer-readable transmission media also include any information delivery media. The term “modulated data signal” means a signal that has one or more of its characteristics set or changed in such a manner as to encode information in the signal. By way of example, and not limitation, computer-readable transmission media include wired media such as a wired network or direct-wired connection, and wireless media such as acoustic, radio frequency (RF), infrared, and other wireless media.

As previously described, hardware elements 710 and computer-readable media 706 are representative of modules, programmable device logic and/or device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 710. The computing device 702 may be configured to implement particular instructions and/or functions corresponding to the software and/or hardware modules. Accordingly, implementation of a module that is executable by the computing device 702 as software may be achieved at least partially in hardware, e.g., through use of computer-readable storage media and/or hardware elements 710 of the processing system 704. The instructions and/or functions may be executable/operable by one or more articles of manufacture (for example, one or more computing devices 702 and/or processing systems 704) to implement techniques, modules, and examples described herein.

The techniques described herein may be supported by various configurations of the computing device 702 and are not limited to the specific examples of the techniques described herein. This functionality may also be implemented all or in part through use of a distributed system, such as over a “cloud” 714 via a platform 716 as described below.

The cloud 714 includes and/or is representative of a platform 716 for resources 718. The platform 716 abstracts underlying functionality of hardware (e.g., servers) and software resources of the cloud 714. The resources 718 may include applications and/or data that can be utilized while computer processing is executed on servers that are remote from the computing device 702. Resources 718 can also include services provided over the Internet and/or through a subscriber network, such as a cellular or Wi-Fi network.

The platform 716 may abstract resources and functions to connect the computing device 702 with other computing devices. The platform 716 may also be scalable to provide a corresponding level of scale to encountered demand for the resources 718 that are implemented via the platform 716. Accordingly, in an interconnected device embodiment, implementation of functionality described herein may be distributed throughout multiple devices of the system 700. For example, the functionality may be implemented in part on the computing device 702 as well as via the platform 716 which may represent a cloud computing environment.

The example systems and methods of the present disclosure overcome various deficiencies of known prior art devices. Other embodiments of the present disclosure will be apparent to those skilled in the art from consideration of the specification and practice of the disclosure contained herein. It is intended that the specification and examples be considered as example only, with a true scope and spirit of the present disclosure being indicated by the following claims.

What is claimed is:

1. A method, comprising:
receiving, from a first sensor located in a care facility, first data associated with the care facility and a patient of the care facility;
determining, based at least in part on the first data and by providing the first data to a machine learning model trained using data labeled with location data associated with an object in a space, a region of focus within the care facility;
causing, by a computing device of the care facility, a second sensor located in the care facility to capture second data associated with the region of focus at a first time;
determining, based at least in part on the first data or the second data, a physiological parameter associated with the patient;
determining, based at least in part on the second data and the first data, a confidence score associated with the physiological parameter; and
recording, based at least in part on the confidence score, the physiological parameter in association with an electronic medical record associated with the patient.

2. The method of claim 1, wherein the first sensor comprises an imaging device, and wherein the first data represents, at least in part, the object within the region of focus.

3. The method of claim 1, wherein the object is disposed at a location within the care facility, the method further comprising:
determining, by the computing device and based on the first data, an environmental attribute of the location; and
selecting, by the computing device and based on the environmental attribute, the second sensor from a plurality of sensors operably connected to the computing device.

4. The method of claim 1, wherein the first data and the second data are characterized by one or more metrics, the method further comprising:
receiving, by the computing device, a request to determine the one or more metrics; and
selecting, by the computing device and based on the request, the second sensor from a plurality of sensors operably connected to the computing device.

5. The method of claim 1, wherein the first data comprises image data, and wherein the method further comprises altering the image data to change at least one of:
a resolution of the image data;
a size of the image data;
a brightness of the image data; or
a contrast of the image data.

6. The method of claim 1, wherein the first sensor or the second sensor includes at least one of:
a RBG camera;
a thermal imaging camera;
a directional microphone;
a LIDAR camera;
a radar sensor;
a proximity sensor;
a weight sensor; or
a physiological parameter sensor.

7. The method of claim 1, wherein at least one of the first sensor and the second sensor are coupled to a moveable platform capable of direction in at least one of an x-axis, a Y-axis, or a Z-axis.

8. The method of claim 7, wherein the first data comprises sequential frames of image data captured over a period of time, and wherein the moveable platform is configured to cause the first sensor to follow the patient within the care facility.

9. The method of claim 1, wherein the first sensor comprises a first sensor type and the second sensor comprises a second sensor type, the second sensor type different from the first sensor type.

10. A system, comprising:
a first sensor located in a care facility comprising a first sensor type;
a second sensor located in the care facility, the second sensor comprising a second sensor type;
one or more processors communicatively coupled to the first sensor and the second sensor; and
a non-transitory, computer-readable media having instruction stored thereon that, when executed by the one or more processors, cause the one or more processors to perform acts comprising:
receiving, from the first sensor located, first data associated with the care facility and a patient of the care facility;
determining, based at least in part on the first data and by providing the first data to a machine learning model trained using data labeled with location data associated with an object in a space, a region of focus within the care facility;
causing the second sensor to capture second data associated with the region of focus at a first time;
determining, based at least in part on the first data or the second data, a physiological parameter associated with the patient;
determining, based at least in part on the first data and the second data, a confidence score associated with the physiological parameter; and
recording, based at least in part on the confidence score, the physiological parameter in association with an electronic medical record associated with the patient.

11. The system of claim 10, wherein the object is disposed at a location within the care facility, the acts further comprising:
determining, based on the first data, an environmental attribute of the location; and
selecting, based on the environmental attribute, the second sensor from a plurality of sensors operably connected to the one or more processors.

12. The system of claim 10, wherein the first data and the second data are characterized by one or more metrics, the acts further comprising:
receiving a request to determine the one or more metrics; and
selecting, based on the request, the second sensor from a plurality of sensors operably connected to a computing device of the care facility.

13. The system of claim 10, wherein the first data comprises image data and the acts further comprise altering the image data to change at least one of:
a resolution of the image data;
a size of the image data;
a brightness of the image data; or
a contrast of the image data.

14. The system of claim 10, wherein the first sensor comprises at least one of:
a RBG camera;
a thermal imaging camera;
a directional microphone;
a LIDAR camera; or
a radar sensor.

15. The system of claim 10, wherein the second sensor comprises at least one of:
a RBG camera;
a thermal imaging camera;
a LIDAR camera;
a radar sensor;
a proximity sensor;
a weight sensor; or
a physiological parameter sensor.

16. The system of claim 10, wherein at least one of the first sensor and the second sensor are coupled to a moveable platform capable of direction in at least one of an x-axis, a Y-axis, or a Z-axis.

17. One or more computer-readable media storing instructions that, when executed by one or more processors, cause the one or more processors to perform operations comprising:
receiving, from a first sensor located in a care facility, first data associated with the care facility and a patient of the care facility;
determining, based at least in part on the first data and by providing the first data to a machine learning model trained using data labeled with location data associated with an object in a space, a region of focus within the care facility;
causing, by a computing device of the care facility, a second sensor located in the care facility to capture second data associated with the region of focus at a first time;
determining, based at least in part on the second data, a physiological parameter associated with the patient;
determining, based at least in part on the second data and the first data, a confidence score associated with the physiological parameter; and
recording, based at least in part on the confidence score, the physiological parameter in association with an electronic medical record associated with the patient.

18. The one or more computer-readable media of claim 17, wherein the object is disposed at a location within the care facility, the operations further comprising:
determining, by the computing device and based on the first data, an environmental attribute of the location; and
selecting, by the computing device and based on the environmental attribute, the second sensor from a plurality of sensors operably connected to the computing device.

19. The one or more computer-readable media of claim 17, wherein the first data and the second data are characterized by one or more metrics, the operations further comprising:
receiving, by the computing device, a request to determine the one or more metrics; and
selecting, by the computing device and based on the request, the second sensor from a plurality of sensors operably connected to the computing device.

20. The one or more computer-readable media of claim 17, wherein at least one of the first sensor or the second sensor are coupled to a moveable platform capable of direction in at least one of an x-axis, a Y-axis, or a Z-axis, and wherein the first data comprises sequential frames of image data captured over a period of time, and wherein the moveable platform is configured to cause the first sensor to follow the patient within the care facility.
